(19) United States
(12) Patent Application Publication
(10) Pub. No.: US 2018/0080072 A1
(43) Pub. Date: Mar. 22, 2018

(54) DETECTION OF NUCLEIC ACID MOLECULES USING NANOPORES AND TAGS

(71) Applicant: Coyote Bioscience Co., Ltd., Beijing (CN)

(72) Inventors: Xiang Li, Beijing (CN); Kun Yang, Shijiazhuang City (CN)

(21) Appl. No.: 15/609,955

(22) Filed: May 31, 2017

Related U.S. Application Data
(63) Continuation of application No. PCT/CN2014/095907, filed on Dec. 31, 2014.

(51) Int. Cl. C12Q 1/68; G01N 33/487

(52) U.S. Cl. CPC C12Q 1/6869; G01N 33/48721

(57) ABSTRACT

The present disclosure provides methods and systems for assaying the presence of a target nucleic acid molecule in a sample having or suspected of having the target nucleic acid molecule. A method of assaying the presence of the target nucleic acid molecule comprises facilitating the flow of the sample through at least one nanopore in a membrane disposed adjacent or in proximity to an electrode that detects a current or current change upon movement of the target nucleic acid molecule through the nanopore. The target nucleic acid molecule, if present, can have a tag coupled at a terminal end thereof that increases a dwell time of the target nucleic acid molecule in the nanopore. The presence of the target nucleic acid molecule in the sample is assayed based on an increase in dwell time of the target nucleic acid molecule from measurements of the current or current change.

### CROSS-REFERENCE

This application is a continuation of PCT International Application No. PCT/CN2014/095907, filed Dec. 31, 2014, which application is herein incorporated by reference in its entirety for all purposes.

### BACKGROUND

A nucleic acid molecule can be amplified, using, for example, thermal cycling based approaches (e.g., polymerase chain reaction (PCR)) or isothermal approaches (e.g., loop-mediated isothermal amplification). Concurrent with or subsequent to amplification of a nucleic acid molecule, amplified products can be detected. This can permit the identification of a nucleic acid sequence of interest such as single nucleotide polymorphisms (SNPs), sequence mutations (including e.g., deletions, insertions, duplication, and translocation), rare nucleic acid molecules/sequences, and other sequences of interest in a sample. Additionally, nucleic acid amplification may be used to prepare a nucleic acid molecule for nucleic acid sequencing.

### SUMMARY

Although there are methods and systems currently available for nucleic acid amplification and sequence identification, various limitations are associated with such methods. Some methods for the identification of a nucleic acid sequence are expensive and may not generate sequence information rapidly enough within a time frame and/or at an accuracy necessary for the intended application. Recognized herein is the need for improved methods for identifying products of nucleic acid amplification reactions, which may enable sequence identification.

The present disclosure provides systems and methods for readily assaying the presence or absence of a target nucleic acid sequence or molecule in a biological sample. In some embodiments, the target nucleic acid molecule can be detected without obtaining a nucleic acid sequence of the target nucleic acid molecule from sequential measurements of signals (e.g., current or change thereof).

An aspect of the present disclosure provides a method for assaying the presence of a target nucleic acid molecule in a sample having or suspected of having the target nucleic acid molecule, the target nucleic acid molecule being coupled to a tag at a terminal end of the target nucleic acid molecule. The method comprises (a) facilitating the flow of the sample through at least one nanopore in a membrane disposed adjacent or in proximity to an electrode that is adapted to detect a current or change thereof upon movement of the target nucleic acid molecule through the at least one nanopore, wherein the movement takes a dwell time that is longer than that of the movement of the target nucleic acid molecule through the at least one nanopore when the target nucleic acid molecule is not coupled to the tag; (b) measuring the current or change thereof with the electrode upon facilitating the flow of the sample through the at least one nanopore; and (c) detecting the target nucleic acid molecule in the sample from the current or change thereof measured in (b), thereby assaying the presence of the target nucleic acid molecule in the sample.

In some embodiments, the tag is a nucleic acid molecule. In some embodiments, the tag is a nucleic acid molecule with at least 5 contiguous nucleotide bases. In some embodiments, the tag is a nucleic acid molecule with at least 10 contiguous nucleotide bases. In some embodiments, the tag is a nucleic acid molecule with at least 20 contiguous nucleotide bases.

In some embodiments, the tag is a molecule comprising a detectable label. For example the tag molecule can be selected from the group consisting of fluorescein amidite (FAM) and hexachloro-fluorescein (HEX). In some embodiments, the tag is a polypeptide. In some embodiments, the tag is not optically detectable.

In some embodiments, the tag is stable at a temperature greater than or equal to 80° C. In some embodiments, the temperature is greater than or equal to about 85° C. In some embodiments, the temperature is greater than or equal to about 90° C. In some embodiments, the temperature is greater than or equal to about 94° C.

In some embodiments, the method further comprises, prior to the step (a) referenced above, the steps of (i) providing a reaction mixture including a biological sample having or suspected of having a template nucleic acid molecule as a precursor of the target nucleic acid molecule, at least one primer that is complementary to the template nucleic acid molecule, and a polymerase, and (ii) subjecting the reaction mixture to a nucleic acid amplification reaction under conditions that yield the target nucleic acid molecule in the sample. In some embodiments, the tag is coupled to the at least one primer. In some embodiments, the sample comprises the target nucleic acid molecule, and wherein the target nucleic acid molecule is a copy among multiple copies as amplification products of the amplification reaction. In some embodiments, the primer is a universal primer, an artificial primer, or a peptide nucleic acid. In some embodiments, the nucleic acid amplification reaction is polymerase chain reaction (PCR). In some embodiments, the nucleic acid amplification reaction is isothermal amplification In some embodiments, the isothermal amplification is loop mediated isothermal amplification (LAMP). In some embodiments, the at least one primer includes at least two primers.

In some embodiments, the step (b) referenced above comprises measuring a change in current, which change is indicative of the presence of the target nucleic acid molecule. In some embodiments, the change in current is a first moment of current with time.

In some embodiments, the current is measured subsequent to facilitating the flow of the sample through the at least one nanopore. In some embodiments, the tag is irreversibly coupled to the target nucleic acid molecule. In some embodiments, the at least one nanopore has a cross-sectional size that is from about 0.5 nanometers (nm) to 30 nm. In some embodiments, the cross-sectional size is from about 2 nm to 15 nm.

In some embodiments, the membrane is a solid state membrane. In some embodiments, the solid state membrane includes a semiconductor or non-metal. In some embodiments, the solid state membrane includes a material selected from the group consisting of carbon, silicon, germanium and gallium arsenide. In some embodiments, the solid state membrane is formed of graphene.

In some embodiments, the membrane is a lipid bilayer. In some embodiments, the at least one nanopore is a pore-forming protein in the membrane. In some embodiments, the pore-forming protein is alpha hemolysin or MspA porin. In some embodiments, the facilitating comprises applying an electrical potential across the at least one nanopore. In some embodiments, the electrical potential is reversible. In some embodiments, the electrical potential is from about 1 V to 10 V relative to a reference.

In some embodiments, the method further comprises applying at least one pulse of an electrical potential across the at least one nanopore to direct the target nucleic acid molecule to and/or through the at least one nanopore. In some embodiments, the at least one nanopore is adjacent or in proximity to an additional electrode. In some embodiments, the target nucleic acid molecule is detected by (i) measuring the current or change thereof upon the flow of the sample through at least one nanopore and (ii) comparing the current or change thereof to a reference.

In some embodiments, the tag increases the dwell time upon interaction of the tag with the at least one nanopore. In some embodiments, the at least one nanopore includes a plurality of nanopores. In some embodiments, the plurality of nanopores are individually addressable. In some embodiments, the target nucleic acid molecule is detected without obtaining a nucleic acid sequence of the target nucleic acid molecule from sequential measurements of the current or change thereof upon the flow of the sample through the at least one nanopore. In some embodiments, the current or change thereof is detected at a dwell time that is indicative of the presence of the target nucleic acid molecule.

In some embodiments, the target nucleic acid molecule includes at least 5 contiguous nucleotide bases. In some embodiments, the target nucleic acid molecule includes at least 10 contiguous nucleotide bases. In some embodiments, the target nucleic acid molecule includes at least 20 contiguous nucleotide bases.

In some embodiments, the target nucleic acid molecule is single stranded. In some embodiments, the target nucleic acid molecule is double stranded. In some embodiments, the target nucleic acid molecule is deoxyribonucleic acid (DNA) or ribonucleic acid (RNA).

Another aspect provides a system for assaying the presence of a target nucleic acid molecule in a sample having or suspected of having the target nucleic acid molecule, the target nucleic acid molecule including at least 5 contiguous nucleotide bases. The system comprises at least one nanopore in a membrane that is disposed adjacent or in proximity to an electrode, wherein the electrode is adapted to detect a current upon flow of a sample through the at least one nanopore; at least one sample holder in fluid communication with the at least one nanopore and adapted to retain the sample; and a computer processor that is operatively coupled to the electrode and programmed to (i) facilitate the flow of the sample from the at least one sample holder through the at least one nanopore, (ii) measure a dwell time of an individual nucleic acid molecule in or through the nanopore, and (iii) identify the individual nucleic acid molecule as the target nucleic acid molecule when the dwell time falls within a reference threshold.

In some embodiments, the computer processor is programmed to measure a first dwell time of the individual nucleic acid molecule in or through the nanopore and identify the individual nucleic acid molecule as the target nucleic acid molecule if the first dwell is longer than a second dwell time of the target nucleic acid molecule in or through the at least one nanopore when the target nucleic acid molecule is not coupled to a tag at a terminal end of the target nucleic acid molecule. In some embodiments, the tag is a nucleic acid molecule. In some embodiments, the tag is a nucleic acid molecule with at least 5 contiguous nucleotide bases. In some embodiments, the tag is a nucleic acid molecule with at least 10 contiguous nucleotide bases. In some embodiments, the tag is a nucleic acid molecule with at least 20 contiguous nucleotide bases. In some embodiments, the tag is a molecule selected from the group consisting of FAM and HEX. In some embodiments, the tag is a polypeptide. In some embodiments, the tag is not optically detectable. In some embodiments, sad tag is stable at a temperature greater than or equal to 80° C.

In some embodiments, the target nucleic acid molecule includes at least 10 contiguous nucleotide bases. In some embodiments, the target nucleic acid molecule includes at least 20 contiguous nucleotide bases.

In some embodiments, the target nucleic acid molecule is single stranded. In some embodiments, the target nucleic acid molecule is double stranded. In some embodiments, the target nucleic acid molecule is DNA or RNA.

In some embodiments, the computer processor is programmed to identify the individual nucleic acid molecule as at least a portion of the target nucleic molecule without obtaining a nucleic acid sequence of the individual nucleic acid molecule.

In some embodiments, the sample has a Mg²⁺ concentration that is less than 1 mole/liter (M).

In some embodiments, the concentration is less than 0.1 M. In some embodiments, the concentration is less than 0.01 M. In some embodiments, the concentration is less than 0.001 M.

In some embodiments, the computer processor is programmed to (a) measure a current or change thereof, and (b) determine the dwell time from the current or change thereof. In some embodiments, the current or change thereof is measured relative to a baseline. In some embodiments, the computer processor is programmed to measure the current or change thereof subsequent to facilitating the flow of the sample through the at least one nanopore. In some embodiments, the computer processor is programmed to determine the dwell time upon comparison of the current or change thereof to a reference.

In some embodiments, the at least one nanopore has a cross-sectional size that is from about 0.5 nanometers (nm) to 30 nm. In some embodiments, the cross-sectional size is from about 2 nm to 15 nm.

In some embodiments, the membrane is a lipid bilayer. In some embodiments, the membrane is a solid state membrane. In some embodiments, the solid state membrane includes a semiconductor or non-metal. In some embodiments, the solid state membrane includes a material selected from the group consisting of carbon, silicon, germanium and gallium arsenide.

In some embodiments, the at least one nanopore is a pore-forming protein in the membrane. In some embodiments, the pore-forming protein is alpha hemolysin or MspA porin.

In some embodiments, the computer processor is programmed to apply an electrical potential across the nanopore. In some embodiments, the electrical potential is reversible. In some embodiments, the electrical potential is from about 1 V to 10 V relative to ground.

In some embodiments, the nanopore is adjacent or in proximity to an additional electrode. In some embodiments, the additional electrode is a reference electrode.

In some embodiments, the at least one nanopore includes a plurality of nanopores. In some embodiments, the plurality of nanopores are individually addressable.

In some embodiments, the at last one nanopore is part of a chip. In some embodiments, the computer processor is part of a circuit having the electrode. In some embodiments, the computer processor is separate from a circuit having the electrode. In some embodiments, the computer processor is an application specific integrated circuit (ASIC). In some embodiments, n the computer processor is part of a mobile electronic device.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a general workflow for the detection of a target nucleic acid molecule.

FIG. 2 shows a nanopore sensor comprising a membrane with a nanopore.

FIG. 3A shows a nanopore sensor comprising a membrane having a nanopore, and a target nucleic acid molecule having a tag at a terminal end thereof adjacent to the membrane; FIG. 3B shows the target nucleic acid threaded through the nanopore of FIG. 3A; FIG. 3C shows the tag interacting with the nanopore or membrane to slow or stop a flow of the target nucleic acid molecule through the nanopore.

FIG. 4 shows a plot of current (i) with time measured by a nanopore sensor.

FIG. 5 shows a computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “membrane,” as used herein, generally refers to a structure that separates at least two volumes of a fluid. Examples of membranes include without limitation solid state membranes and lipid bilayers. A membrane may be an organic membrane, such as a lipid bilayer, or a synthetic membrane, such as a membrane formed of a solid state material (e.g., semiconductor, metal, semi-metal or non-metal) or polymeric material.

The term “nanopore,” as used herein, generally refers to a pore, channel or passage formed or otherwise provided in a membrane. The nanopore may be disposed adjacent or in proximity to a sensing circuit or an electrode coupled to a sensing circuit, such as, for example, a complementary metal-oxide semiconductor (CMOS) or field effect transistor (FET) circuit. In some examples, a nanopore has a characteristic size (e.g., cross-section, width or diameter) on the order of 0.1 nanometers (nm) to about 1000 nm. Some nanopores are proteins. Alpha hemolysin is an example of a protein nanopore.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof including but not limited to peptide nucleic acid (PNA). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double stranded. A nucleic acid may comprise one or more modified nucleotides, such as methylated nucleotides and nucleotide analogs.

The term “polymerase,” as used herein, generally refers to any enzyme capable of catalyzing a polymerization reaction. Examples of polymerases include, without limitation, a nucleic acid polymerase, a transcriptase or a ligase. A polymerase can be a polymerization enzyme.

The term “tag,” as used herein, generally refers to any atomic or molecular species that couples (e.g., attaches) to a nucleic acid molecule at a terminal end thereof. The tag can directly couple to the terminal end of the nucleic acid molecule or indirectly through a linker. A tag can be a nucleic acid molecule (e.g., polynucleotide), a polypeptide, a protein (e.g., enzyme), a polymeric material or other moiety that can interact with a nanopore or membrane to slow the progression of a nucleic acid molecule through the nanopore. For example, the tag can be a nucleic acid molecule with at least 5, 10, or 20 contiguous nucleotide bases. The tag can be a mass tag. The tag can be a fluorescent dye or a fluorophore. Examples of tags include, without limitation, proteins, fluorescein amidite (FAM), hexachloro-fluorescein (HEX), biotin, tetrachlorofluorescein (TET), tetramethylrhodamine (TAMRA), cyanine dyes (e.g., Cy3 or Cy5), sulforhodamine 101 acid chloride (Texas Red), black hole quenchers (BHQ) and 4-(dimethylaminoazo)benzene-4-carboxylic acid (Dabcyl). The tag may not be optically detectable.

The tag can have a cross-section size that is larger than that of a nanopore. In some cases, a tag interacts with a nanopore or membrane to slow the progression of a nucleic acid molecule through the nanopore. The interaction can be reversible or irreversible.

The term “subject,” as used herein, generally refers to an animal or other organism, such as a mammalian species (e.g., human), avian (e.g., bird) species, or plant. Mammals include, but are not limited to, murines, simians, humans, farm animals, sport animals, and pets. A subject can be an individual that has or is suspected of having a disease or a pre-disposition to the disease, or an individual that is in need of therapy or suspected of needing therapy. A subject can be a patient.

The term “sample,” as used herein, generally refers to any sample containing or suspected of containing a nucleic acid molecule. For example, a subject sample can be a biological sample containing one or more nucleic acid molecules. The biological sample can be obtained (e.g., extracted or isolated) from a bodily sample of a subject that can be selected from blood (e.g., whole blood), plasma, serum, urine, saliva, mucosal excretions, sputum, stool and tears. The bodily sample can be a fluid or tissue sample (e.g., skin sample) of the subject. In some examples, the sample is obtained from a cell-free bodily fluid of the subject, such as whole blood. In such instance, the sample can include cell-free DNA and/or cell-free RNA. In some other examples, the sample is an environmental sample (e.g., soil, waste, ambient air and etc.), industrial sample (e.g., samples from any industrial processes), and food samples (e.g., dairy products, vegetable products, and meat products).

The term “genome variation,” as used herein, generally refers to a variant or polymorphism in a nucleic acid sample or genome of a subject. Examples of variants include single nucleotide polymorphism, single nucleotide variant, insertion, deletion, substitution, repeat, variable length tandem repeat, flanking sequence, structural variant, transversion, rearrangement and copy number variation.

### Assaying the Presence of a Target Nucleic Acid Molecule

An aspect of the present disclosure provides methods and systems for assaying the presence of a target nucleic acid molecule in a sample. The target nucleic acid molecule can have a nucleic acid sequence of interest for an intended application, including without limitation, species identification, environmental testing, forensic analysis, and general research and disease characterization.

A sensor can be used to detect the presence of the target nucleic acid molecule in the sample. The sensor can have an array of one or more nanopores that are configured to detect current or a change in current with time. The target nucleic acid molecule can be detected by measuring current (C) or current change (or first moment of current with time, dC/dt) with time, and in some cases comparing such measurement to a reference (or baseline).

The sample can include one or more molecules, at least some of which can be the target nucleic molecule. A dwell time (or residence time) of any molecule in or through the nanopore can be indicative of the presence of the target nucleic molecule in the sample. In some situations, the target nucleic acid molecule has a detectable dwell time in the nanopore, which can be greater than other molecules in the sample. By measuring current or current change with time and determining dwell times, the target nucleic acid molecule, if present, can be detected in the sample.

The dwell time of the target nucleic acid molecule can be increased using a tag that couples (e.g., attaches) to the target nucleic acid molecule at a terminal end of the target nucleic acid molecule. The tag can slow the flow of the target nucleic acid molecule through the nanopore. The tag can be a protein, such as an enzyme, a polynucleotide, or other moiety that can interact with a nanopore or membrane to reduce or stop the flow of a target nucleic acid molecule through the nanopore. The tag can increase the dwell time upon interaction of the tag with the nanopore or membrane. For example, the tag can have a cross-sectional size that is larger than a cross-sectional size of the nanopore. The target nucleic acid molecule having the tag coupled thereto is directed through the nanopore and is unable to flow through the nanopore. This reduces or stops the flow of the target nucleic acid molecule through the nanopore.

The interaction between the tag and the nanopore or membrane can be reversible such that, upon the application of a stimulus, the interaction can be broken or otherwise removed and the target nucleic acid molecule can exit the nanopore. Such stimulus can be a voltage, such as a voltage pulse (e.g., a 10V pulse), or a pressure drop.

The target nucleic acid molecule can be deoxyribonucleic acid (DNA), ribonucleic acid (RNA), or a variant thereof. The target nucleic acid sample can be processed, such as by fragmenting the target nucleic acid sample into fragments. The target nucleic acid molecule can be single stranded or double stranded.

The target nucleic acid molecule can include contiguous nucleotides. In some examples, the target nucleic acid molecule includes at least 5, 10, 30, 40, 50, 100, 200, 300, 400, 500, or 1000 nucleotides.

The target nucleic acid molecule can be an amplification product of a template nucleic acid molecule in the sample. In some cases, the target nucleic acid molecule can be detected by obtaining a biological sample from a subject and subjecting the sample to nucleic acid amplification to amplify at least a portion of the template nucleic acid molecule. Nucleic acid amplification can be performed under conditions that are selected to amplify the template nucleic acid molecule or a portion thereof if a nucleic acid sequence of interest is present. If the nucleic acid sequence of interest is present, nucleic acid amplification can yield one or more amplified nucleic acid products. Such products can include the target nucleic acid molecule.

The template nucleic acid molecule can be DNA, RNA, or a variant thereof. The template nucleic acid sample can be processed, such as by fragmenting the template nucleic acid sample into fragments. The template nucleic acid molecule can be single stranded or double stranded.

Once the sample has been subjected to nucleic acid amplification, the target nucleic acid molecule can be detected. This can be performed using sensors described elsewhere herein. The target nucleic acid molecule can be detected without nucleic acid sequencing, such as obtaining a nucleic acid sequence of the target nucleic acid molecule or other nucleic acid molecule in the sample. For example, the presence of the target nucleic acid molecule can be determined without sequencing by synthesis techniques (e.g., Illumina, Pacific Biosciences of California, Genia or Ion Torrent). The presence of the target nucleic acid molecule can be determined without sequential measurements of a signal(s) (e.g., an optical signal or current) that is indicative of at least 1, 2, 3, 4 or 5 nucleotides of the target nucleic acid molecule.

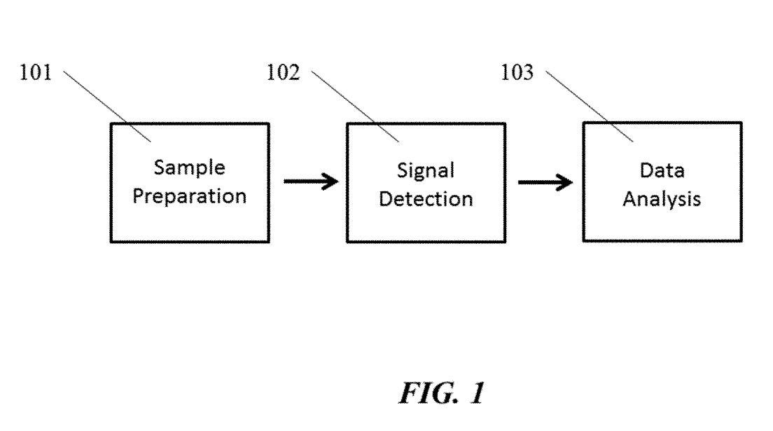

FIG. 1 shows a workflow for sample processing. In a first operation 101, a biological sample is prepared for detection. The biological sample can be obtained from a bodily fluid of a subject, for example, and a nucleic acid molecule can be isolated from the bodily fluid. The nucleic acid molecule can be a template nucleic acid molecule for subsequent analysis. In some cases, the nucleic acid molecule is processed to yield the template nucleic acid molecule, such as fragmented to yield multiple template nucleic acid molecules.

The template nucleic acid molecule can be subsequently subjected to nucleic acid amplification conditions to amplify (i.e., generate one or more copies) the template nucleic acid molecule. An amplification product of the template nucleic acid molecule can be a target nucleic acid molecule for subsequent analysis.

In some cases, a reaction mixture comprising a biological sample having or suspected of having the template nucleic acid molecule as a precursor of the target nucleic acid molecule is provided. The reaction mixture can also include at least one primer that is complementary to the template nucleic acid molecule and a polymerase. The at least one primer can include 1, 2, 3, 4, 5, 6, 7, 8, 9, 10, 20, 30, 40, or 50 primers. Each primer can have a sequence that is selected for a particular type of analysis, such as detecting a given disease or genome variation in a subject.

A primer can be coupled to a tag or multiple tags. Such coupling can be by way of direct attachment or attachment through a linker. For example the primer can be attached to a biotin, FAM or HEX moiety. This can permit amplification of the template nucleic acid molecule with the primer to yield a target nucleic acid molecule as an amplification product. The target nucleic acid molecule can have the tag incorporated at a terminal end of the target nucleic acid molecule. As an alternative, however, the primer is not coupled to the tag, and the tag is provided at a subsequent point in time. In some examples, the primer is an artificial primer, such as locked nucleic acid (LNA), or a peptide nucleic acid (PNA). The primer can be a universal primer.

Next, the reaction mixture can be subjected to a nucleic acid amplification reaction under conditions that yield the target nucleic acid molecule in the sample. The target nucleic acid molecule can be a copy among multiple copies of the template nucleic acid molecule, which are amplification products of the nucleic acid amplification reaction.

The reaction mixture can include reagents necessary to complete nucleic acid amplification (e.g., DNA amplification, RNA amplification), with non-limiting examples of such reagents including primer sets having specificity for target RNA or target DNA, DNA produced from reverse transcription of RNA, a DNA polymerase, a reverse transcriptase (e.g., for reverse transcription of RNA), suitable buffers (including zwitterionic buffers), co-factors (e.g., divalent and monovalent cations), dNTPs, and other enzymes (e.g., uracil-DNA glycosylase (UNG)), etc). In some cases, reaction mixtures can also comprise one or more reporter agents. The reaction mixture can also include an enzyme that is suitable to facilitate nucleic acid amplification, such as a polymerizing enzyme (also “polymerase” herein). The polymerase can be a DNA polymerase for amplifying DNA. Any suitable DNA polymerase may be used, including commercially available DNA polymerases. The DNA polymerase can be capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases.

In some cases, a DNA sample can be generated from an RNA sample. This can be achieved using reverse transcriptase, which can include an enzyme that is capable of incorporating nucleotides to a strand of DNA, when bound to an RNA template. Any suitable reverse transcriptase may be used. Non-limiting examples of reverse transcriptases include HIV-1 reverse transcriptase, M-MLV reverse transcriptase, AMV reverse transcriptase, telomerase reverse transcriptase, and variants, modified products and derivatives thereof.

Nucleic acid amplification reaction can include one or more primer extension reactions to generate amplified product(s). In PCR, for example, a primer extension reaction can include a cycle of incubating a reaction mixture at a denaturation temperature for a denaturation duration and incubating a reaction mixture at an elongation temperature for an elongation duration. Denaturation temperatures may vary depending upon, for example, the particular biological sample analyzed, the particular source of target nucleic acid (e.g., viral particle, bacteria) in the biological sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be at least about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

As an alternative, in isothermal amplification, the temperature can be fixed (i.e., not cycled), and amplification product(s) can be generated using a primer set and a polymerase with high strand displacement activity in addition to a replication activity. An example of a polymerase that may be suitable for use in isothermal amplification is Bst polymerase. The temperature can be fixed between about 50° C. and 80° C., or 60° C. and 65° C. In loop mediated isothermal amplification (LAMP), for example, a template nucleic acid molecule can be amplified using a polymerase and a primer set having at least 2, 3, 4, or 5 primers.

During or subsequent to nucleic acid amplification, a tag can be provided to the reaction mixture. For example, a primer used in nucleic acid amplification can be coupled to a tag, such as at a terminal end. For example, the tag can be directly attached to a 5′ end of the primer by direct attachment or through a linker. In an example, the 5′ end of the primer is attached to a biotin, FAM or HEX moiety. This can permit amplification of the template nucleic acid molecule with the primer to yield a target nucleic acid molecule as an amplification product, the target nucleic acid molecule having the tag incorporated at a terminal end of the target nucleic acid molecule. The tag can permit detection of the target nucleic acid molecule using a nanopore sensor of the present disclosure. In some cases, the primer is coupled to multiple tags, such as at least 2, 3, 4 or 5 tags.

With continued reference to FIG. 1, in a second operation 102, subsequent to subjecting the template nucleic acid molecule to nucleic acid amplification, the presence of the target nucleic molecule as an amplification product can be determined. This can be achieved by detecting one or more signals that are indicative of the presence of the target nucleic acid molecule, such as dwell time of the target nucleic acid molecule upon measurements of current or a change in current with time using sensors described elsewhere herein. Next, in a third operation 103, the one or more signals are analyzed to determine whether the target nucleic acid molecule is present or not present. The one or more signals can also be analyzed to determine a relative quantity of the target nucleic molecule.

The amplification of the template nucleic acid molecule and detection of the target nucleic acid molecule can be performed in the same system, such as vessel. In some cases, the system is a tube that is configured for nucleic acid amplification, such as an eppendorf PCR tube. As an alternative, amplification and detection are in separate systems. For example, amplification is performed in an eppendorf PCR tube and detection is performed in a separate chip having a nanopore sensor.

### Nanopore Sensors

Another aspect of the present disclosure provides nanopore sensors for detecting a target nucleic acid molecule. A nanopore sensor can include an array of one or more nanopores in a membrane. Each nanopore can be disposed adjacent to a measurement electrode that is configured to detect a current or current change with time, in some cases with reference to a reference electrode.

The array can include at least 1, 2, 3, 4, 5, 6, 7, 8, 9, 10, 100, 200, 300, 400, 500, 1000, 10000, 100000 or 1000000 sensors. Each sensor can include at least 1, 2, 3, 4 or 5 nanopores. Each sensor can be individually addressable. The density of nanopores can be at least about 100, 200, 300, 400, 500, 600, 700, 800, 900, 1000, 10000, 100000, 10⁶, 10⁷, 10⁸, 10⁹, 10¹⁰, or 10¹¹ nanopores per square millimeter (mm²).

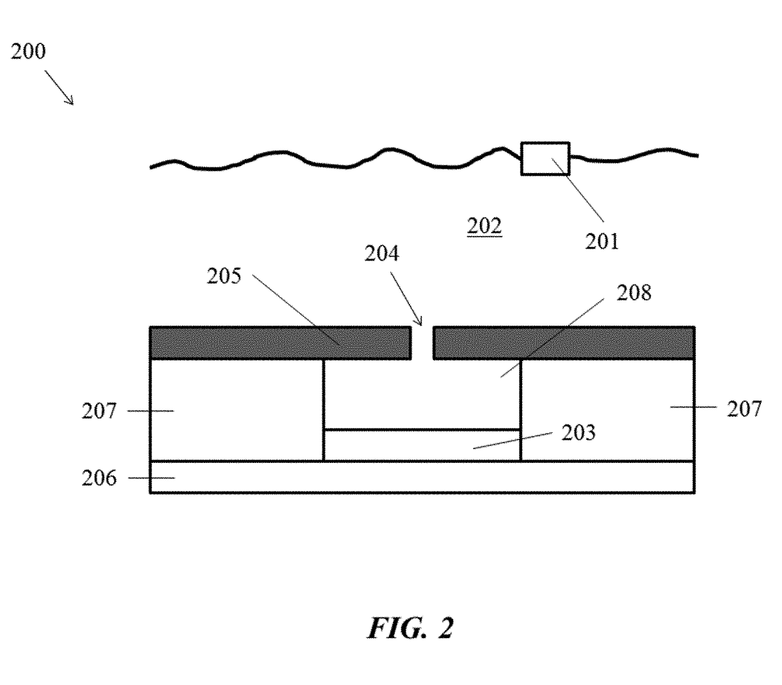

FIG. 2 shows a nanopore sensor 200 comprising a first electrode 201 in contact with a conductive solution 202 (e.g., salt solution). The sensor 200 comprises a second electrode 203 near, adjacent, or in proximity to a nanopore 204 in a membrane 205. The second electrode 203 is adjacent to a circuit element 206 having electrical circuitry for signal (e.g., current or current change) measurements. The membrane 205 is adjacent to a chamber 208 (e.g., well) that is at least partially defined by a wall 207. The wall 207 can be formed of a semiconductor, such as silicon oxide or aluminum oxide (e.g., SiO₂). As an alternative, the wall 207 is formed of a polymeric material. In some examples, the wall 207 is part of a tube that is usable for nucleic acid amplification.

The nanopore sensor 200 can be in a container (e.g., tube) that is configured for nucleic acid amplification, such as an eppendorf PCR tube. The container can include a top chamber for nucleic acid amplification of a template nucleic acid molecule and a bottom chamber for subsequent detection of the target nucleic acid molecule. The container can be disposable and/or reusable.

As an alternative, the nanopore sensor 200 can be part of a chip that includes a sample holder. The sample holder can contain a sample having or suspected of having a target nucleic acid molecule. The chip can have onboard electronics (e.g., a computer processor) for signal detection and processing. As an alternative, the onboard electronics can be off-chip, such as in a computer system adjacent to the chip and in communication with the chip. The chip can be disposable and/or reusable. The circuit element 206 can include electrical current flow paths that bring the nanopore sensor 200 in communication with the computer system.

For example, the nanopore sensor 200 is part of a container or chip that is insertable into and removable from a reader (not shown). The reader can include a computer processor that permits the detection of the target nucleic acid molecule in a sample having or suspected of having the target nucleic acid molecule. As alternative, the computer processor is in a computer system that is separate from and in communication with the reader. The reader can include a fluid flow system (e.g., pumps and actuators) that directs the sample to the nanopore sensor.

The nanopore sensor 200 can include an array of at least 1, 2, 3, 4, 5, 6, 7, 8, 9, 10, 100, 200, 300, 400, 500, 1000, 10000, 100000 or 1000000 sensors. Each sensor can include at least 1, 2, 3, 4 or 5 nanopores.

The membrane 205 can be a solid state membrane. The membrane 205 can be formed of a semiconductor or non-metal. In some examples, the membrane 205 is formed of a material selected from carbon, silicon, germanium and gallium arsenide. For example, the membrane 205 can be formed of graphene.

As an alternative, the membrane 205 can be a lipid bilayer. The lipid bilayer can include two layers of lipid molecules. The lipid bilayer can include phospholipids with hydrophilic head and two hydrophobic tails each. When exposed to water, such phospholipids can arrange themselves into a two-layered sheet (a bilayer) with all of their tails pointing toward the center of the sheet. The center of this bilayer can contain little to no water and exclude molecules. An outer surface of the lipid bilayer can be hydrophilic while an inner portion of the lipid bilayer can be hydrophobic.

The nanopore 204 can be a hole providing a channel through the membrane 205. As an alternative, the nanopore 204 can be a pore-forming protein in the membrane 205. Such alternative can be used in situations in which the membrane 205 is a lipid bilayer. The pore-forming protein can be alpha hemolysin or MspA porin.

The nanopore 204 can have a cross-sectional size that permits fluid flow through the nanopore 204. The cross-sectional size can permit flow of a nucleic acid sample through the nanopore 204. The cross-sectional size can be from about 0.5 nanometer (nm) to 30 nm, or 1 nm to 20 nm, 2 nm to 15 nm, 3 nm to 10 nm, or 2.5 nm to 3.4 nm.

The nanopore 204 can have various shapes and sizes. For example, the nanopore 204 can have a rectangular shape, hour glass shape, concave shape, convex shape, a conical shape, or partial shapes or combinations thereof. The nanopore 204 can have a length that spans the membrane 205. In some cases, the nanopore 204 has a length from about 10 nm to 5000 nm, or 20 nm to 1000 nm, or 30 nm to 1000 nm, and the membrane 205 has a thickness from about 10 nm to 5000 nm, or 20 nm to 1000 nm, or 30 nm to 1000 nm. The length of the nanopore 204 can be the same as the thickness of the membrane 205, or different. For example, the nanopore 204 can span at least about 50%, 60%, 70%, or 80% the thickness of the membrane 205.

The membrane 205 includes a trans side and a cis side. The cis side is adjacent to the first electrode 201. During use, a target nucleic acid molecule having a tag coupled to a terminal end is directed from the cis side to the trans side of the membrane 205. The cis side is opposite from the trans side. The tag can have a size that slows or stops the flow of the target nucleic acid molecule through the nanopore, or be configured to interact with the nanopore 204 or membrane 205 to slow or stop the flow of the target nucleic acid molecule through the nanopore 204. For example, the tag is larger than a cross-section size of the nanopore 204. As another example, the tag interacts with a portion of a channel or lip of the nanopore 204 to slow or stop the flow of the target nucleic acid molecule through the nanopore 204.

The solution 202 can have an electrolyte. The electrolyte can include one or more salts, such as NaCl, KCl, or AgCl. The solution 202 can have a salt concentration that permits the detection of a current using the first electrode 201 and second electrode 203. In an example, the concentration can be from about 0.1 mole/liter (M) to 10 M, or 2 M to 8 M. As another example, the concentration can be from 0.1 mM to 10 mM, or 0.5 mM to 5 mM.

The solution 202 can include a buffer for PCR. For example, the solution 202 can include 50 mM to 200 mM Tris-HCl (e.g., 100 mM Tris-HCl), 200 mM to 1000 mM KCl (e.g., 500 mM KCl), and 0.5 mM to 5 mM MgCl₂.

The first electrode 201 and second electrode 203 can be formed of one or more metals. In some cases, the first electrode 201 and second electrode 203 are formed of Au, Ag or Pt. For example, the first electrode 201 is formed of Pt and the second electrode 203 is formed of Ag. As an alternative, the first electrode 201 is formed of Pt and the second electrode 203 is formed of AgCl.

In some cases, the second electrode 203 is formed of a material that permits the electrochemical depletion of the electrode 203 during detection. For example, the second electrode 203 can be formed of AgCl. During operation of the sensor 200, AgCl→Ag⁺+Cl⁻. This can be reversed by applying an inverse electrical potential to the second electrode 203 to deposit AgCl onto the second electrode 203, thereby reversing depletion.

In some cases, the sensor 200 is operated by application of a direct current (DC) voltage to the second electrode 203 relative to the first electrode 201. The voltage can range from 0.5 volts (V) to 20 V, or 1 V to 10 V. In such DC operation, the voltage can be reversed (i.e., V→−V→V). As an alternative, the sensor is operated by application of an alternating current (AC) voltage to the second electrode 203 relative to the first electrode 201. The voltage can range from 0.5 V to 20 V, or 1 V to 10 V.

During operation of the sensor 200, an electric field can be provided across the nanopore 204 upon the application of a voltage between the first electrode 201 and the second electrode 203. The electric field can be configured to direct a target nucleic acid molecule in the solution 202 towards the nanopore 204. The electric field can aid the target nucleic acid molecule to approach and move through the nanopore 204. As an alternative or in addition to, a pressure drop can be provided across the nanopore 204, which can aid the target nucleic acid molecule to approach and move through the nanopore 204. In some cases, a pressure-derived force exceeds the opposing voltage-derived force. The motion of the target nucleic acid molecule can be regulated using the combination of a pressure drop and an electric field. For instance, the movement of the target nucleic acid molecule can be slowed upon the application of an electric field from the trans side to the cis side of the membrane 205 while applying a pressure drop from the cis side to the trans side. As an alternative, the movement of the target nucleic acid molecule can be accelerated upon the application of an electric field from the cis side to the trans side of the membrane 205 while applying a pressure drop from the cis side to the trans side.

In some cases, the pressure-derived and voltage-derived forces are balanced to regulate (e.g., increase or decrease) a translocation time (or dwell time) of a target nucleic acid molecule through the nanopore 204. The charge can be deduced from the balance of forces on the molecule via the relationship qE=Fmech, where ‘E’ is the electric field in the nanopore 204, which can be a function of the voltage applied between the electrodes 201 and 203, and Fmech is the sum of the mechanical forces on the target nucleic acid molecule from the applied pressure and/or the fluid flow through the nanopore 204.

During use of the sensor 200, the circuit 206 provides an electrical potential across the first electrode 201 and the second electrode 203. An electrolyte in the solution 202 can transport ions in the solution 202 through the nanopore 204. During use, the second electrode 203 can undergo an oxidation reaction to yield ions of the second electrode 203 in the solution 202, which can be directed through the nanopore towards the first electrode 201. A reduction reaction can occur at the first electrode 201 using ions in the solution 202.

Upon the flow of the solution 202 through the nanopore 204, a measurable current can be detected using the first electrode 201 and the second electrode 203. The current can change with a change in flow rate of a flow through the nanopore 204. For example, upon obstruction of the nanopore 204 with a target nucleic acid molecule, the flow rate can change, which can lead to change in current measured by the first electrode 201 and the second electrode 203. Such change in current can be related to the size and time of the obstruction. A molecule that obstructs the nanopore 204 for a longer period of time can effect a current change for a longer period of time, which can be proportional to the dwell time of the molecule in the nanopore. The intensity of the change in current can be directly related to the size of the obstruction. For example, a larger molecule in or flowing through the nanopore 204 can effect a larger change in current as compared to a smaller molecule in or flowing through the nanopore 204.

If the target nucleic acid molecule is present in solution, it can be prepared in a manner to have a tag coupled thereto that increases the dwell time of the target nucleic acid molecule in the nanopore 204. Such increase in dwell time can impart a change in current (C) or current change (dC/dt) over time, which can be detected by the electrodes 201 and 203.

The circuit 206 can reverse the direction of the electrical potential across the first electrode 201 and the second electrode 203. This can aid in reversing any depletion of the second electrode 203. For example, to deposit ions from the solution 202 on the second electrode, the electrical potential across the first electrode 201 and the second electrode 203 can be reversed, which can provide a reduction reaction at the second electrode 203 (e.g., Ag⁺+Cl⁻→AgCl).

Nanopore sensors of the present disclosure can be used to detect a target nucleic acid molecule. Such detection can be facilitated by increasing a dwell time of the target nucleic acid molecule in, adjacent, or in proximity to a nanopore of a nanopore sensor, thereby affecting fluid flow through the nanopore. This can generate a measurable current or change in current at electrodes of the nanopore sensor. The dwell time of the target nucleic acid molecule can be increased using a tag coupled to a terminal end of the target nucleic acid molecule.

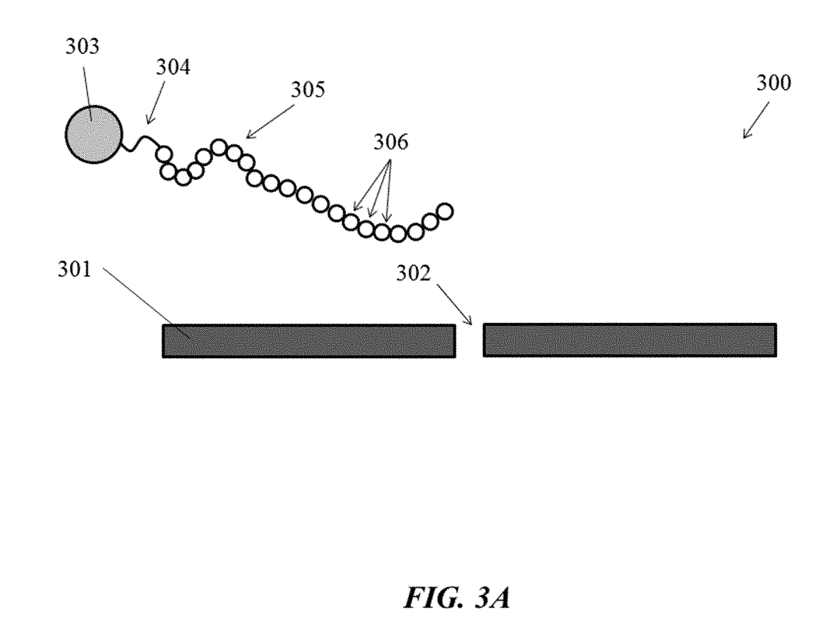

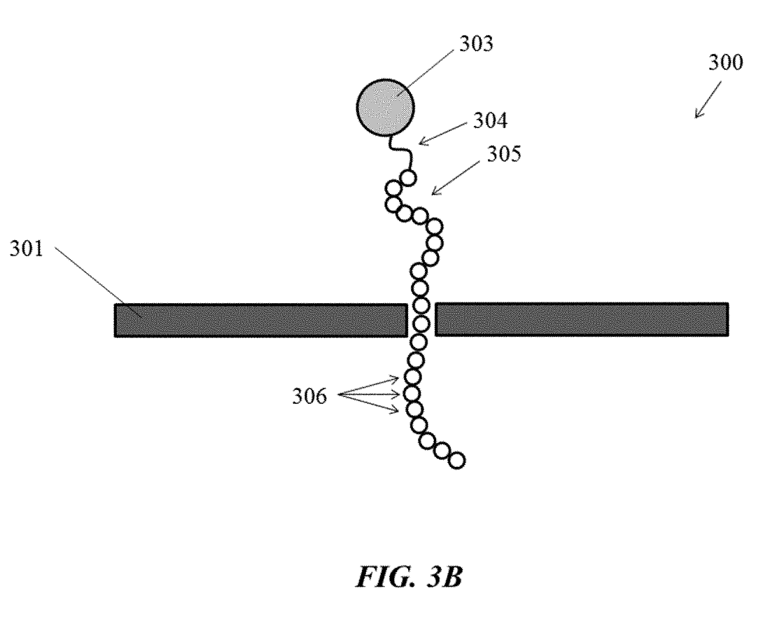

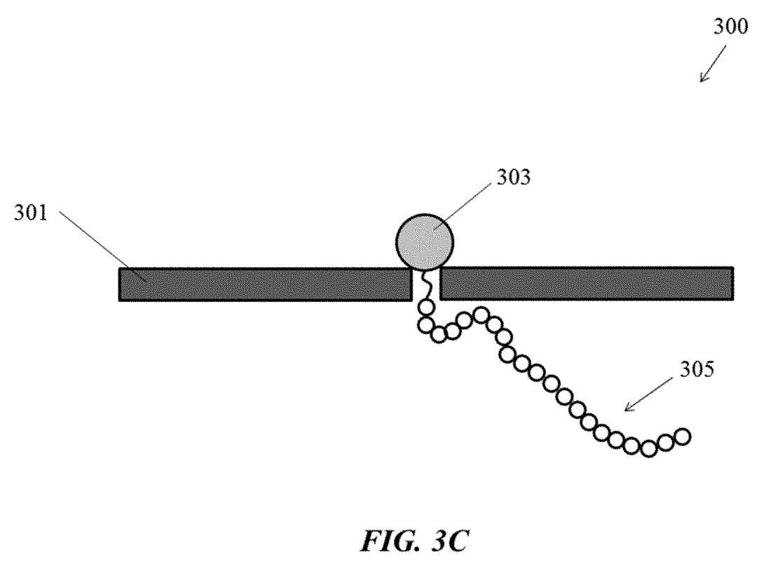

FIGS. 3A-3C schematically illustrate the detection of a target nucleic acid molecule using a nanopore sensor 300. With reference to FIG. 3A, the nanopore sensor 300 includes a membrane 301 having a nanopore 302. A tag 303 is attached to a linker 304 which is attached to a target nucleic acid molecule 305. The target nucleic acid molecule 305 is disposed in proximity to the membrane 301 at a cis side of the membrane 301. The target nucleic acid molecule 305 includes contiguous nucleic acid subunits 306 (or nucleotides). The nanopore sensor 300 includes electrodes (not shown), which can be as described elsewhere herein. The target nucleic acid molecule 305 can be directed to the nanopore 302 upon the application of an electrical potential (V) between the electrodes, which can provide an electric field that directs the target nucleic acid molecule 305 to the nanopore 302.

The tag 303 can be a moiety that is selected to interact with the nanopore 302 in a manner that reduces the flow rate of the target nucleic acid mole protein through the nanopore 302. For example, the tag 303 is a biotin, FAM or HEX moiety. Conditions of the solution can be selected such that the activity of the moiety is not substantially affected. The tag 303 can be stable at a temperature greater than or equal to 80° C., 85° C., 90° C., or 94° C. In some cases, the tag 303 is stably coupled to the target nucleic acid molecule 305 at a temperature greater than or equal to 80° C., 85° C., 90° C., or 94° C.

A solution having the tag 303, linker 304 and target nucleic acid molecule 305 can have conditions selected such that the activity of the tag 303 and linker 304 is not substantially affected. For example, the tag 303 and linker 304 may not have reduced or substantially diminished activity at amplification conditions.

In some examples, the tag 303 is a protein, such as an enzyme. The enzyme may be a polymerase or a molecular motor. The enzyme can have reduced activity or is not enzymatically active. Conditions of the solution can be selected such that the enzyme has reduced activity or is not enzymatically active. The conditions can be selected from the group consisting of salt (or ion) concentration and temperature of the sample.

The linker 304 can be a molecule that includes one or more nucleic acid or amino acid moieties, such as a polynucleotide or polypeptide. The linker 304 can be a polymer. In some cases, the linker 304 is a polymer such as a peptide, nucleic acid, polyethylene glycol (PEG). The linker 304 can be of any suitable length. For example, the linker 304 can have a length that is at least about 1 nm, 5 nm, or 10 nm. The linker 304 can be rigid or flexible.

The nanopore 302 can have a cross-sectional size (e.g., diameter) from about 0.5 nanometer (nm) to 30 nm, or 1 nm to 20 nm, 2 nm to 15 nm, 3 nm to 10 nm, or 2.5 nm to 3.4 nm. The tag 303 can have a cross-sectional size (e.g., diameter or effective diameter) that is larger than a cross-sectional size of the nanopore 302. As an alternative, the tag 303 has a cross-sectional size that is smaller than the nanopore 302, but the tag 303 is configured to interact with the nanopore 302 to reduce or stop the flow of the target nucleic acid molecule 305 through the nanopore 302. For example, the tag can be smaller than the nanopore 302 but include charge carrying groups that interact with charge carrying groups in the membrane 301 or nanopore 302 that are similarly polarized (e.g., both positively charged or negatively charged) such that an interaction between such charge carrying groups provides a repulsive interaction that reduces or stops the flow of the target nucleic acid molecule 305 through the nanopore 302.

In FIG. 3B, the target nucleic acid molecule 305 is directed through the nanopore 302, such as upon the application of the electrical potential between the electrodes. Because the cross-sectional size of the target nucleic acid molecule 305 is less than the cross-sectional size of the nanopore 302, the target nucleic acid molecule 305 flows through the nanopore 302 from the cis side of the membrane 301 to the trans side of the membrane (or vice versa, in some cases). In FIG. 3C, the tag 303 interacts with the membrane 301 or nanopore 302. Such interaction can slow or stop the flow of the target nucleic acid molecule 305 through the nanopore 302, which increases the dwell time of the target nucleic acid molecule 305 in the nanopore 302. The increased dwell time can be detected by the electrodes as a measureable change in current (C) or current change (dC/dt) with time. With the aid of the tag 303, the target nucleic acid molecule 305 may get stuck in the nanopore 302, which can generate a measurable current that can enable the detection of the target nucleic acid molecule 305 from other nucleic acid molecules that are not coupled to tags.

The target nucleic acid molecule 305 can be detected without obtaining a nucleic acid sequence of the target nucleic acid molecule 305 from sequential measurements of the current or change thereof upon the flow of the sample through the nanopore 302. The current or change thereof can be detected at a dwell time that is indicative of the presence of the target nucleic acid molecule 305. For example, a current measured from 1 millisecond (ms) and 10 ms can be indicative of the presence of the target nucleic acid molecule 305, while a current measured at less than 1 ms can be indicative of other molecules or species in solution that may not be target nucleic acid molecule 305.

The target nucleic acid molecule 305 can be detected by measuring a current or change thereof upon the flow of a sample having or suspected of having the target nucleic acid molecule 305 through the nanopore 302. The measured current or change thereof can be compared to a reference (e.g., baseline current or current change). Any difference with respect to such reference as a function of time can be indicative of the presence of the target nucleic acid molecule 305.

Subsequent to detecting the target nucleic acid molecule 305, a stimulus can be provided to remove the target nucleic acid molecule 305 from the nanopore 302. The stimulus can be a pressure pulse, heat pulse, voltage pulse, the application of a sheer force, or a combination thereof. In some cases, the stimulus breaks the interaction between the tag 303 and the membrane 301 or nanopore 302. For instance, the stimulus breaks the interaction between the tag 303 and the target nucleic acid molecule 305, for example, by breaking the linker 304. As an alternative, the stimulus is a reversal of the direction of flow, such as upon the application of a negative pressure drop or voltage. This induces the target nucleic acid molecule 305 to reverse flow direction and exit the nanopore 302 from the trans side to the cis side.

In some examples, the stimulus is a voltage pulse supplied by between the electrodes of the nanopore sensor. The voltage pulse can include a voltage from about 0.5 V to 20 V, or 1 V to 10 V, and supplied for at time period from about 500 nanoseconds (ns) and 2 ms, or 500 ns and 1 ms. For example, the voltage pulse is an electrical potential of 5 V for a time period of about 1 ms. In some cases, the pulse duration is less than or equal to about 5 ms, 4 ms, 3 ms, 2 ms, or 1 ms. The voltage can have a polarity opposite from the polarity used to direct the target nucleic acid molecule 305 into the nanopore 302.

The stimulus can be applied to the membrane 301 and/or the nanopore 302. The stimulus can be directed to the membrane 301 and/or the nanopore 302 under conditions such that the membrane 301 and/or the nanopore 302 is not disrupted.

For example, if a voltage of V (e.g., 0.5 mV) was used to direct the target nucleic acid molecule 305 to the nanopore 302 along a direction leading from the cis side to the trans side of the membrane 301, a voltage of −V can be used to direct the target nucleic acid molecule 305 out of the nanopore from the trans side to the cis side of the membrane 301.

As another example, if a pressure drop ΔP (e.g., 1 atm) across the nanopore 302 was used to direct the target nucleic acid molecule 305 to the nanopore 302 along a direction leading from the cis side to the trans side of the membrane 301, a pressure drop of −ΔP can be used to direct the target nucleic acid molecule 305 out of the nanopore from the trans side to the cis side of the membrane 301.

Once the target nucleic acid molecule 305 is removed from the nanopore 302, the nanopore 302 can be used to detect the presence of another target nucleic acid molecule in solution. For example, a pressure drop (e.g., ΔP) and/or voltage (V) can be supplied across the nanopore 302 to direct another target nucleic acid molecule 305 have a tag 303 coupled thereto into the nanopore 302.

The target nucleic acid molecule 305 can be coupled to one tag or multiple tags. In some cases, multiple tags (e.g., 2, 3, 4, or 5 tags) can provide for a dwell time of the target nucleic acid molecule 305 in the nanopore 302 that can provide for higher detection sensitivity (e.g., greater than 90%). Multiple tags can be directly coupled to the target nucleic acid molecule 305, or coupled indirectly through one or more linkers.

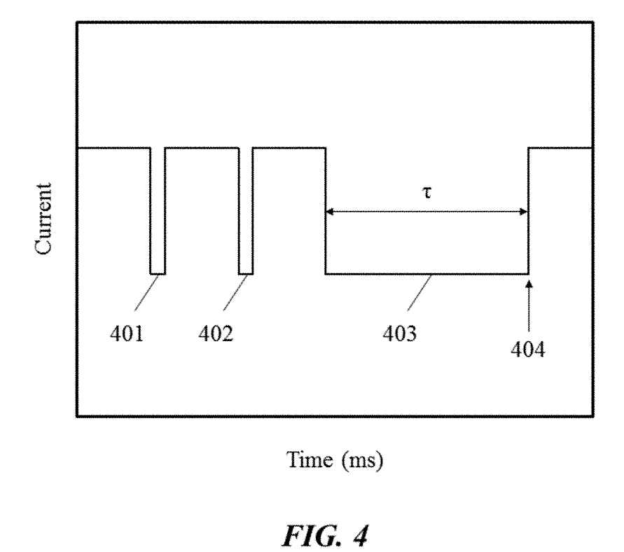

FIG. 4 shows an example plot of current measurement (y axis) with time (x axis, milliseconds (ms)) using a nanopore sensor of the present disclosure. The nanopore sensor includes a membrane with a nanopore. Over the detection time period, the flow of a solution through the nanopore is slowed or otherwise disrupted three times, yielding current signals 401, 402 and 403. Each change in current 401-403 has a dwell time (t). Comparing the dwell time to a reference can lead to the determination that the current signal 403 is associated with a target nucleic acid molecule and the signals 401 and 402 are not associated with the target nucleic acid molecule. The determination can be made by measuring a change in current (e.g., AC versus time or dC/dt versus time) that is indicative of the presence of the target nucleic acid molecule stopped or stalled in the nanopore by a tag. For example, from reference measurements (i.e., with a sample having a known target nucleic acid molecule), any dwell time greater than or equal to 5 ms can be attributed to a target nucleic acid molecule. The signals 401 and 402 have dwell times of about 1 ms, and the signal 403 has a dwell time that is greater than 5 ms.

The signal 403 can persist until a stimulus is applied to the nanopore and/or the membrane to remove the target nucleic acid molecule from the nanopore. In the illustrated example, a voltage pulse is applied to the nanopore and/or the membrane at time 404.

The signals 401 and 402 that are not associated with the target nucleic acid molecule can each persist for a given period of time independent of the stimulus. The signal 403 can persist until the stimulus is applied at time 404.

The amplitude of the signals 401, 402 and 403 can be the same or different. In some cases, the amplitude of the signal 403 is different than the amplitude of the signals 401 and 402.

The nanopore sensor can measure current continuously or periodically. In some cases, the nanopore sensor measures current subsequent to facilitating the flow of a solution with a sample having or suspected of having the target nucleic acid molecule through the nanopore.

### Methods for Forming Nanopores

Nanopores of the present disclosure can be formed via a variety of methods. For instance, an array of one or more nanopores can be formed using photolithography in which a pattern of one or more holes is defined in a photoresist (e.g., poly(methyl methacrylate)) and transferred to a substrate (e.g., silicon substrate) using photolithography, which can include exposing the pattern of one or more holes to an anisotropic chemical etchant.

In some cases, a substrate is provided and a photoresist layer is provided adjacent to the substrate. The photoresist layer can be formed of, for example, poly(methyl methacrylate) (PMMA), poly(methyl glutarimide) (PMGI), phenol formaldehyde resin, or an expoxy-based negative photoresist (e.g., SU-8). The photoresist can be developed upon exposure to light, such as ultraviolet (UV) light

Next, the photoresist can be exposed to a pattern of electromagnetic radiation or particles (e.g., light or electron beam) to define a hole in the photoresist that exposes the substrate. The exposure to light can cause a chemical change that allows some of the photoresist to be removed by a wash solution, leaving the hole. A positive photoresist can become soluble in the wash solution when exposed, while in a negative photoresist, unexposed regions are soluble in the wash solution. Next, the hole can be exposed to a chemical etchant. The chemical etchant can provide anisotropic etching. For example, the chemical etchant can be potassium hydroxide (KOH). In some cases, a focused ion beam and/or a time buffered oxide etch (BOE) can be used to provide fine etching, such as removal of residual oxide.

The substrate can be a semiconductor or polymer substrate. For example, the substrate can be formed of silicon, germanium, carbon (e.g., graphene), or gallium arsenide, or an oxide or nitride thereof. As an example, the substrate is formed of silicon, silicon oxide or silicon nitride. As another example, the substrate can be formed of a metal, such as copper, nickel, or aluminum. The substrate can have a thickness from about 10 nm to 5000 nm, or 20 nm to 1000 nm, or 30 nm to 1000 nm. In an example, the substrate has a thickness from about 50 nm to 150 nm.

Nanopores formed according to methods provided herein can have various electrical conductances. For example, a nanopore having a cross-sectional size from about 5 nm to 15 nm can have an electrical conductance from about 20 nano Siemens (nS) to 150 nS, 50 nS to 120 nS, or 60 nS and 110 nS. Such conductance may be measured with respect to the flow of an electrolyte, such as KCl.

### Computer Control Systems

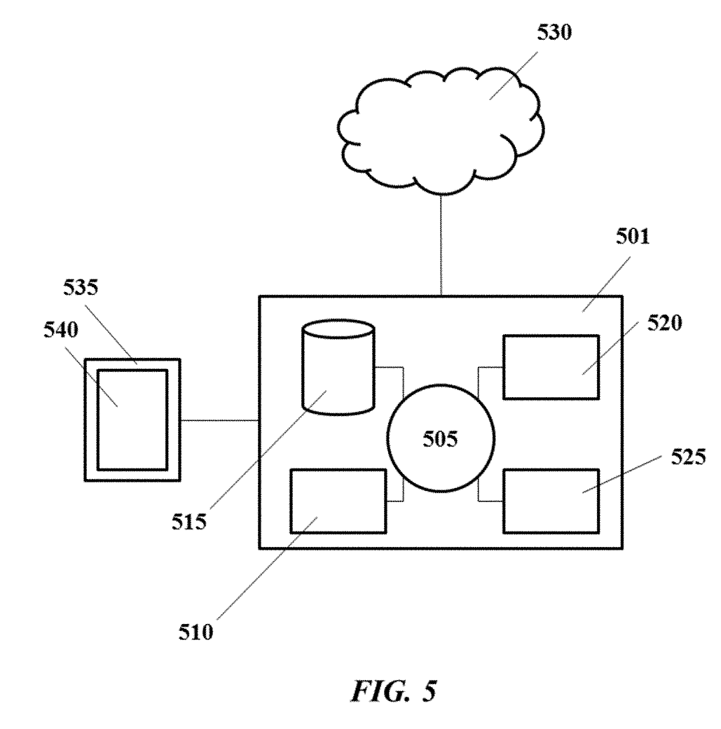

The present disclosure provides computer control systems that are programmed to implement methods of the disclosure. FIG. 5 shows a computer system 501 that is programmed or otherwise configured to detect the presence of a target nucleic acid sample in solution. The computer system 501 can regulate various aspects of nanopore sensor of the present disclosure, such as, for example, detecting current or current change with time. The computer system 501 can be in communication with a nanopore sensor, which can be part of a chip. The computer system 501 can be stationary or mobile. In some examples, the computer system 501 is part of a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user (e.g., service provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

A machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, signals from a nanopore sensor with time. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505.

### Example 1

A semiconductor substrate (e.g., silicon) is irradiated with energetic particles in a processing chamber. The energetic particles can be argon ions (e.g., Ar+). At least one nanopore is generated in the semiconductor substrate using photolithography and etching. For example, a mask can be provided adjacent to the semiconductor and locations of the mask corresponding to the nanopore is exposed and the mask in such locations is removed to expose a portion of the semiconductor substrate. The exposed portion of the semiconductor substrate is contacted with an etching solution (e.g., mixture of HF and HNO₃) to etch the nanopore into the semiconductor substrate. An etch block layer in the semiconductor substrate can terminate the etching. The semiconductor substrate with nanopore can be provided adjacent to electrodes to provide the nanopore sensor.

The nanopore sensor can be provided in an effendorf PCR tube, including chamber for PCR reaction and for detection. The semiconductor with the nanopore can be a membrane that separates two wells, a cis well and a trans well. Reagents used for nucleic acid amplification (e.g., isothermal amplification) are added to the cis well. Reagents for nucleic acid amplification can include a PCR buffer, primer, DNA polymerase and template nucleic acid sample. LAMP and endonuclease can be conducted at a temperature of about 65° C. to generate a double stranded target nucleic acid molecule as an amplification product of the template nucleic acid molecule. Next, the endonuclease is covalently cross-linked with the nanopore. A voltage is applied between the cis and trans wells and a current is measured with the nanopore sensor.

The voltage between the wells (across the cis and trans sides) induces the negatively charged target nucleic acid molecule to enter and electrophorese through the nanopore. The target nucleic acid molecule has a tag at a terminal end thereof that increases the dwell time of the target nucleic acid molecule in the nanopore. Based on the increased dwell time, the presence of the target nucleic acid molecule is identified. The target nucleic acid molecule with the tag is distinguishable from other nucleic acid molecules not having a tag based on the dwell time.

### Example 2

A thin film of 2 μm wet thermal silicon oxide and 100 nm low pressure chemical vapor deposition (LPCVD) low-stress (silicon rich) silicon nitride are deposited on 500 μm thick P-doped (100) Si wafers of 1-20 ohm·cm resistivity. Freestanding 20 μm membranes are formed by anisotropic KOH (33%, 80° C.) etching of wafers in which the thin films has been removed in a photolithographically patterned region by reactive ion etching. A focused ion beam (Micrion 9500) is used to remove about 1.5 μm of silicon oxide in a 1 μm square area in the center of the freestanding membrane. A subsequent BOE removes about 600 nm of the remaining oxide, leaving a 2 μm free-standing mini-membrane of silicon nitride in the center of the freestanding oxide/nitride membrane. The nitride film is about 80 nm thick after processing in KOH and BOE, as measured by ellipsometry and cross-sectional transmission electron microscopy (TEM). A focused 200 keV electron beam from a JEOL 2010F field-emission TEM (JEOL USA, Peabody, Mass.) is used to form roughly hourglass-shaped nanopores in the center of the nitride minimembrane. Nanopore diameters are about 10 nm.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for assaying the presence of a target nucleic acid molecule in a sample having or suspected of having said target nucleic acid molecule, said target nucleic acid molecule being coupled to a tag at a terminal end of said target nucleic acid molecule, the method comprising:
(a) facilitating the flow of said sample through at least one nanopore in a membrane disposed adjacent or in proximity to an electrode that is adapted to detect a current or change thereof upon movement of said target nucleic acid molecule through said at least one nanopore, wherein said movement takes a dwell time that is longer than that of the movement of said target nucleic acid molecule through said at least one nanopore when said target nucleic acid molecule is not coupled to said tag;
(b) measuring said current or change thereof with said electrode upon facilitating the flow of said sample through said at least one nanopore; and
(c) detecting said target nucleic acid molecule in said sample from said current or change thereof measured in (b), thereby assaying the presence of said target nucleic acid molecule in said sample.

2. The method of claim 1, wherein said tag is a nucleic acid molecule or a polypeptide.

3. The method of claim 2, wherein said tag is a nucleic acid molecule with at least 5 contiguous nucleotide bases.

4.-7. (canceled)

8. The method of claim 1, wherein said tag is not optically detectable.

9. The method of claim 1, wherein said tag is stable at a temperature greater than or equal to 80° C.

10.-12. (canceled)

13. The method of claim 1, further comprising, prior to (a), (i) providing a reaction mixture including a biological sample having or suspected of having a template nucleic acid molecule as a precursor of said target nucleic acid molecule, at least one primer that is complementary to said template nucleic acid molecule, and a polymerase, and (ii) subjecting said reaction mixture to a nucleic acid amplification reaction under conditions that yield said target nucleic acid molecule in said sample.

14. The method of claim 13, wherein said tag is coupled to said at least one primer.

15.-20. (canceled)

21. The method of claim 1, wherein (b) comprises measuring a change in current, which change is indicative of the presence of said target nucleic acid molecule.

22. The method of claim 1, wherein said change thereof is a first moment of current with time.

23.-33. (canceled)

34. The method of claim 1, wherein said facilitating comprises applying an electrical potential across said at least one nanopore.

35. (canceled)

36. (canceled)

37. The method of claim 1, further comprising applying at least one pulse of an electrical potential across said at least one nanopore to direct said target nucleic acid molecule to and/or through said at least one nanopore.

38. The method of claim 1, wherein said at least one nanopore is adjacent or in proximity to an additional electrode.

39. (canceled)

40. The method of claim 1, wherein said tag increases said dwell time upon interaction of said tag with said at least one nanopore.

41. (canceled)

42. (canceled)

43. The method of claim 1, wherein said target nucleic acid molecule is detected without obtaining a nucleic acid sequence of said target nucleic acid molecule from sequential measurements of said current or change thereof upon the flow of said sample through said at least one nanopore.

44. The method of claim 1, wherein said current or change thereof is detected at a dwell time that is indicative of the presence of said target nucleic acid molecule.

45.-48. (canceled)

49. A system for assaying the presence of a target nucleic acid molecule in a sample having or suspected of having said target nucleic acid molecule, the target nucleic acid molecule including at least 5 contiguous nucleotide bases, the system comprising:
at least one nanopore in a membrane that is disposed adjacent or in proximity to an electrode, wherein said electrode is adapted to detect a current upon flow of a sample through said at least one nanopore;
at least one sample holder in fluid communication with said at least one nanopore and adapted to retain said sample; and
a computer processor that is operatively coupled to said electrode and programmed to (i) facilitate the flow of said sample from said at least one sample holder through said at least one nanopore, (ii) measure a dwell time of an individual nucleic acid molecule in or through said nanopore, and (iii) identify said individual nucleic acid molecule as said target nucleic acid molecule when said dwell time falls within a reference threshold.

50. The system of claim 49, wherein said computer processor is programmed to measure a first dwell time of said individual nucleic acid molecule in or through said nanopore and identify said individual nucleic acid molecule as said target nucleic acid molecule if said first dwell is longer than a second dwell time of said target nucleic acid molecule in or through said at least one nanopore when said target nucleic acid molecule is not coupled to a tag at a terminal end of said target nucleic acid molecule.

51.-61. (canceled)

62. The system of claim 49, wherein said computer processor is programmed to identify said individual nucleic acid molecule as at least a portion of said target nucleic molecule without obtaining a nucleic acid sequence of said individual nucleic acid molecule.

63.-66. (canceled)

67. The system of claim 49, wherein said computer processor is programmed to (a) measure a current or change thereof, and (b) determine said dwell time from said current or change thereof.

68. (canceled)

69. (canceled)

70. The system of claim 67, wherein said computer processor is programmed to determine said dwell time upon comparison of said current or change thereof to a reference.

71.-78. (canceled)

79. The system of claim 49, wherein said computer processor is programmed to apply an electrical potential across said nanopore.

80. (canceled)

81. (canceled)

82. The system of claim 49, wherein said nanopore is adjacent or in proximity to an additional electrode.

83.-85. (canceled)

86. The system of claim 49, wherein said at last one nanopore is part of a chip.

87. The system of claim 49, wherein said computer processor is part of a circuit having said electrode.

88. (canceled)

89. (canceled)

90. The system of claim 49, wherein said computer processor is part of a mobile electronic device.
